(19) United States
(12) Patent Application Publication
(10) Pub. No.: US 2024/0005151 A1
(43) Pub. Date: Jan. 4, 2024

(54) DEEP LEARNING TECHNIQUES FOR ELASTICITY IMAGING

(71) Applicant: THE REGENTS OF THE UNIVERSITY OF CALIFORNIA, Oakland, CA (US)

(72) Inventors: GRACE XIANG GU, ORCHARD LAKE, MI (US); CHUN-TEH CHEN, BERKELEY, CA (US)

(21) Appl. No.: 18/253,901

(22) PCT Filed: Nov. 22, 2021
(86) PCT No.: PCT/US2021/060362

§ 371 Date: May 22, 2023

Related U.S. Application Data
(60) Provisional application No. 63/117,554, filed on Nov. 24, 2020.

(51) Int. Cl. G06N 3/08

(52) U.S. Cl. CPC G06N 3/08

(57) ABSTRACT

A method of predicting elasticity of a solid includes receiving a data set comprised of position data and corresponding strain data for points on a solid at a deep neural network (DNN), producing a predicted stress distribution, applying convolutional filters to the predicted stress distribution to produce residual force maps, and predicting an elasticity distribution of the solid by iteratively using the residual force maps and an equilibrium condition until the predicted elasticity distribution satisfies the equilibrium condition, producing the final elasticity distribution.

### CROSS REFERENCE TO RELATED APPLICATION

This application claims priority to and the benefit of U.S. Provisional Application No. 63/117,554 filed Nov. 24, 2020, which is incorporated herein by reference in its entirety.

### TECHNICAL FIELD

This disclosure relates to elastography, more particular to using deep learning to determine hidden physical properties of solids.

### BACKGROUND

Being able to image the material property distribution of solids non-invasively has a broad range of applications in materials science, biomechanical engineering, and clinical diagnosis. For instance, as various disease progresses, the elasticity, quantified as the Young's modulus or shear modulus, of human cells, tissues, and organs may be altered significantly. Palpation, such as breast self-examination, utilizes the difference between the elasticity of healthy and cancerous tissues to distinguish them. Elasticity imaging, known as elastography, is an emerging method to qualitatively image the elasticity distribution of an inhomogeneous body.

A long-standing goal of elastography is to provide alternative methods of clinical palpation for reliable tumor diagnosis. The displacement distribution of a body under externally applied forces, or displacements, can be acquired by a variety of imaging techniques such as ultrasound, magnetic resonance, and digital image correlation. A strain distribution, determined by the gradient of a displacement distribution, can be computed or approximated from measured displacements.

If the strain and stress distributions of a body are both known, the elasticity distribution can be computed using the constitutive elasticity equations, Hooke's law. However, there is currently no technique that can measure the stress distribution of a body in vivo. Therefore, in elastography, the stress distribution of a body is commonly assumed to be uniform and a measured strain distribution can be interpreted as a relative elasticity distribution. This approach is referred to as strain-based elastography and has the advantage of being easy to implement. The uniform stress assumption in this approach, however, is inaccurate for an inhomogeneous body.

The stress field of a body can be distorted significantly near a hole, inclusion, or wherever the elasticity varies. This phenomenon, known as stress concentration, is of great interest in industry and academia. Though the strain-based elastography has been deployed on many commercial ultrasound diagnostic-imaging devices, the elasticity distribution predicted based on this method is prone to inaccuracies. To mitigate this misinterpretation, a research field focusing on solving an inverse problem associated with elasticity imaging has been extensively investigated for decades. In this approach, referred to as model-based elastography, the elasticity distribution of a body may be, in principle, recovered by modeling its elastic behavior and solving an inverse problem.

Inverse problems arise in many scientific and engineering fields and are typically difficult to solve by conventional approaches. Much progress towards artificial intelligence (AI) and machine learning (ML) have been made and provided novel directions to solve these inverse problems. For instance, ML techniques were applied to solve inverse problems in materials design, fluid mechanics, and PDEs (partial differential equations). In principle, supervised learning may work for this inverse problem if the number of possible elasticity distributions can be reduced. A simple way to do so is, for instance, to consider a uniform soft body containing a few hard inclusions and to constrain the shapes, sizes, locations, and elastic moduli of these hard inclusions. However, adding such artificial constraints to the inverse problem may limit the application of the model in practice.

### BRIEF DESCRIPTION OF THE DRAWINGS

FIG. 1 shows a flowchart of an embodiment of a method of predicting elasticity in solids.

FIGS. 2A-B show a graphical representation of the stresses and the resulting kernels for a representative solid.

FIGS. 3A-C show graphical representations of physical constraints on a method of predicting elasticity for a solid.

FIGS. 4A-C show graphical representations of the effects of noise in measurements on a method of predicting elasticity for a solid.

FIGS. 5A-C show graphical representations of the effects of missing data in measurements on a method of predicting elasticity for a solid.

FIGS. 6A-D show graphical representations of super-resolution in elasticity imaging.

FIGS. 7A-C show graphical representations of experimental validation of the elasticity network.

FIG. 8 shows an embodiment of a computer system used to predict elasticity in solids.

### DETAILED DESCRIPTION

The embodiments here formulate an inverse problem in elasticity to determine the elasticity distribution of a body from a given displacement, or strain, distribution. It is an inverse problem since a typical forward problem in elasticity is to determine the displacements of a body from a given elasticity distribution.

To tackle this inverse problem, the dominant approaches in the literature are based on minimizing the difference between measured and simulated displacements. However, these approaches are computationally expensive as they require many iterations and each iteration requires solving a forward problem and conducting sensitivity analysis using the finite element method (FEM).

It is possible to solve this inverse problem directly. In some approaches, known as direct approaches, measurements are considered as coefficients in the partial differential equations (PDEs) of equilibrium. Though these direct approaches have computationally efficiency, they may perform poorly when measurements contain large strain gradients or noise, or the elasticity distribution is not smooth. Moreover, the error from noise tends to propagate along the integration path when solving the PDEs and may cause inaccurate predictions. Due to these limitations, most model-based elastography methods were only applied to solve simple problems in elasticity imaging such as a uniform soft body containing a few hard inclusions.

The embodiments here have taken the possibility of applying ML techniques to solve the inverse problem in elasticity. To obtain useful information from an elasticity image, the number of pixels, the resolution, is typically on the order of 10³ to 10⁵. Due to the high-dimensional input and output spaces, the hidden correlation between the strain and elasticity distributions of a body is difficult to be captured by supervised learning using labeled data.

The embodiments here introduce a de novo elastography method to learn the hidden elasticity of a body with a deep neural network. The embodiments involve a new method not using supervised learning with labeled data; the theory of elasticity does the supervising, essentially with unlabeled data. The constitutive elasticity equations and equilibrium equations for solving the inverse problem are encoded in the neural network. The embodiments provide a new method of a general-purpose approach for elastography. The embodiments do not impose artificial constraints on possible elasticity distributions. They show that the proposed method can accurately reconstruct the elasticity distribution of a body from a given strain distribution.

The proposed method is robust when it comes to noisy and missing measurements. Moreover, the proposed method can predict a probable elasticity distribution for areas without data based on the elasticity distribution in nearby regions. This ability can be used to generate super-resolution elasticity images. The embodiments demonstrate that the proposed method not only can learn the hidden elasticity but also can decrypt the hidden physics of the inverse problem when strain and elasticity distributions are both given. The unique features of the proposed method make it a promising tool for a broad range of elastography applications, including those involving human tissue and the displacement distribution of a body under externally applied forces, or displacements, can be acquired by a variety of imaging techniques such as ultrasound, magnetic resonance, and digital image correlation.

The embodiments here involve a method uses a deep neural network (DNN) to learn the hidden elasticity of a body from a given strain distribution. As used here the term “deep neural network” means a neural network having at least three layers. The DNN will generally have inputs, weights, a bias, threshold or other constraint, and an output. In the embodiments here the dataset comprises position points on an image of a solid and strain measurements for each position. However, the input is only the position points. The strain measurements are used to update the weights being applied. The threshold or constraint here is the elasticity constitutive relation and an equilibrium equation or condition. The output comprises a predicted elasticity distribution that can be used to analyze the image, possibly in a medical diagnostic environment.

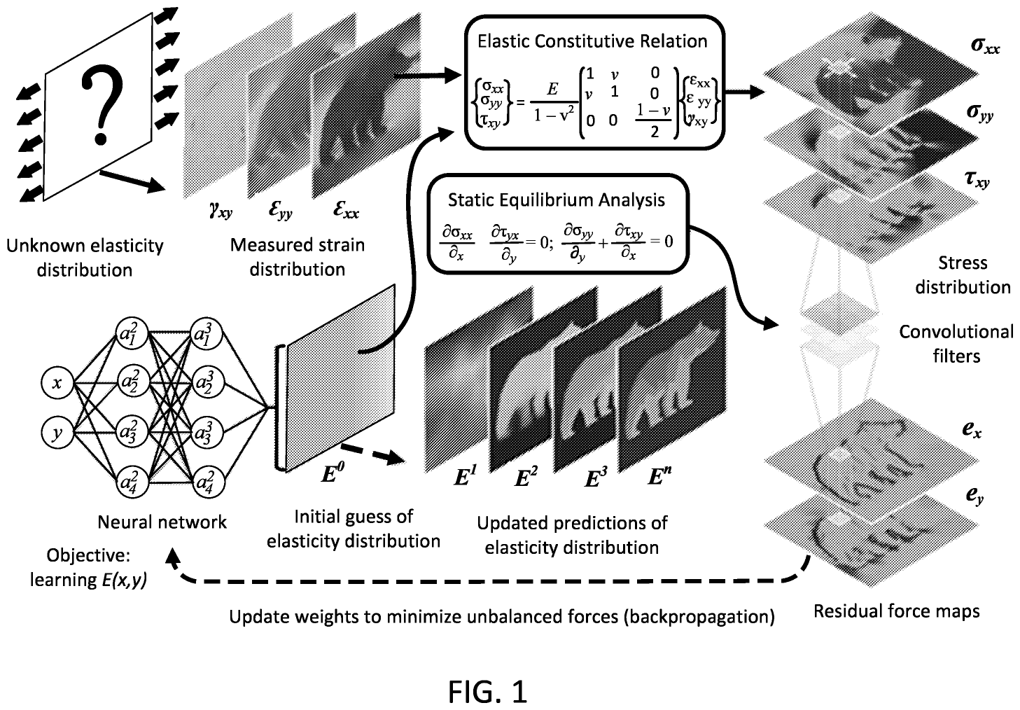

The flowchart of the proposed method is shown in FIG. 1. The DNN function, f_{θ}, with learning parameters θ is not trained from labeled data. The DNN is never shown the correct elasticity distribution. By contrast, it is supervised by the theory of elasticity, allowing it to escape the performance ceiling imposed by labeled data. The elastic constitutive relation (σ=C_{ε}) and equilibrium equation (∇σ=0) for solving the inverse problem in elasticity are encoded in the DNN as prior knowledge. Biological tissues are mainly composed of water, and they are nearly incompressible. Here, all material points in a body of interest are assumed to be linear, isotropic, and incompressible. The elasticity at a material point can be described by either Young's modulus E or shear modulus G. Without loss of generality, Young's modulus E is used to quantify the elasticity in these embodiments. The body is assumed to be a thin plate, and therefore the nonzero stress components are σₓₓ, σ_{yy}, and τ_{xy} (plane stress state). The information of position p and strain ε at all material points is converted to a data set. The stress σ at each material point is calculated by the encoded elastic constitutive relation based on the measured strain ε and predicted elasticity E as discussed below. The measured strain c for each point results from experiments or a simulation. As will be discussed below, the measured strain may occur on a lower resolution.

In FIG. 1, the DNN f_{θ} takes the information of position p at each material point as an input and outputs its elasticity, E=f_{θ}(p). The information of strain E at each material point is not involved in the forward propagation for predicting the elasticity but is used in the back propagation for updating the weights θ. Using the elastic constitutive relation, the stress at each material point is calculated based on the measured strain and predicted elasticity. When the entire data set is passed forward through the DNN, a predicted stress distribution n is generated.

A predicted stress distribution θ includes three stress images σₓₓ, σ_{yy}, τ_{xy}) shown on the top left of FIG. 1. Before training, these strain images are unlikely to satisfy the conditions for equilibrium, as the initial elasticity distribution E is generated by random initialization of weights θ and biases in the DNN. To evaluate how close the predicted stress distribution θ is from equilibrium, the stress images are passed forward through a convolutional layer in the DNN. Unlike other convolutional neural networks, in which the kernels need to be learned from labeled data, the kernels in the convolutional layer of the embodiments are encoded in such a way that the convolution operation is used to evaluate the conditions for equilibrium, discussed below.

Residual, unbalanced, force maps are generated after the convolution operation. The training procedure minimizes the norms of the residual forces with an additional physical constraint, discussed below, and updates the weights θ using backpropagation. Consequently, the predicted elasticity distribution E is updated and then used in the next iteration of training. This training procedure repeats until the predicted elasticity distribution E is converged, meaning that the predicted stress distribution θ satisfies the equilibrium conditions such that the predicted stress distribution θ is in equilibrium.

The deep-learning framework in the embodiments is similar in spirit to the so-called physics-informed deep learning, in which physical laws are encoded into the loss function. Most physics-informed models used automatic differentiation to solve the PDEs (partial differential equations) in physics. However, automatic differentiation may not be appropriate for the inverse problem in these embodiments. As mentioned, measured strains naturally contain noise, and differentiating the strains can amplify the noise significantly. Moreover, this approach requires the elasticity distribution of a body to be differentiable, which is often not true in practice. Instead of using automatic differentiation, the embodiments use a convolution operation to solve the PDEs for equilibrium.

The process considers a small cube with sides of length h. The equilibrium conditions for the cube can be expressed as:

σₓₓ(x+h,y)−σₓₓ(x,y)+τ_{yx}(x,y+h)−τ_{yx}(x,y)=0 (1)

σ_{yy}(x,y+h)−σ_{yy}(X,y)+τ_{xy}(X+h,y)−T_{xy}(X,y)=0

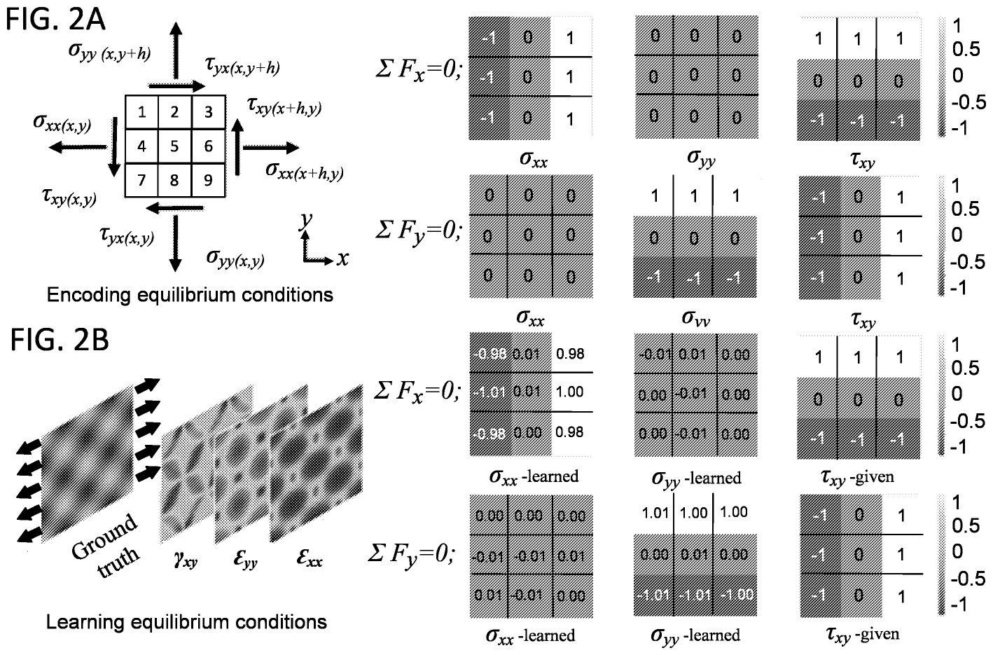

Let the cube contain 3-by-3 material points as shown in FIG. 2A. The equilibrium conditions for the cube can be expressed in terms of the stresses at the material points:

$${\sum }_{a = 1}^{3} {\sum }_{b = 1}^{3} {w}_{xx} ( a , b ) {σ}_{xx} ( a , b ) + {w}_{yy} ( a , b ) {σ}_{yy} ( a , b ) + {w}_{xy} ( a , b ) {τ}_{xy} ( a , b ) = 0 \qquad ( 2 )$$

where wₓₓ, w_{yy}, and w_{xy}, are the convolution kernels for σₓₓ, σ_{yy}, and τ_{xy}, respectively. By choosing proper sets of values for the kernels, Equation (2) can be used to describe the equilibrium conditions for the cube. These sets of values are then encoded in the kernels in FIG. 2A to describe the equilibrium conditions in the x-direction and y-direction. These kernels are used as “equilibrium detectors” in the DNN and the convolution operation generates residual force maps, shown in FIG. 1, in which each element is calculated by:

$$e ( i , j ) = {\sum }_{a = 1}^{3} {\sum }_{b = 1}^{3} {w}_{xx} ( a , b ) {σ}_{xx} ( i + a - 1 , j + b - 1 ) + {w}_{yy} ( a , b ) {σ}_{yy} ( i + a - 1 , j + b - 1 ) + {w}_{xy} ( a , b ) {τ}_{xy} ( i + a - 1 , j + b - 1 ) ) ht \qquad ( 3 )$$

where t is the thickness of the cube. Here, the embodiments show that the conditions for equilibrium can be encoded in the DNN as domain knowledge for solving the inverse problem. Now, the discussion considers the possibility of learning this domain knowledge from labeled data. To test this idea, an inhomogeneous body is modeled by the finite element method (FEM) with a 128×128 mesh. The elasticity field of the body is defined by a two-dimensional sinusoidal function,

$$E ( x , y ) = 0.45 ( 1 + cos ( \frac{4 π x}{L} ) cos ( \frac{4 π y}{L} ) ) + 0.1 , \\ x ∈ [ 0 , L ] , y ∈ [ 0 , L ] \qquad ( 4 )$$

where L is the length of the body in both x-direction and y-direction. The unit is set to be megapascal (MPa). Consequently, the maximum and minimum Young's moduli are 1 MPa and 0.1 MPa, respectively. This “sinusoidal” model is subjected to externally applied displacements along the x-direction on the boundary. An average normal strain (εₓₓ) of 1% is introduced by the applied displacements. The details of the finite element analysis are summarized below.

The elasticity and strain distributions of the sinusoidal model are shown in FIG. 2B. To learn the conditions for equilibrium, the elasticity and strain distributions are both fed into the DNN, and the loss function is defined as the mean absolute error (MAE) of the residual forces. From Eq. 3, it can be seen that the kernels (wₓₓ, w_{yy}, and w_{xy}) cannot be uniquely determined by minimizing the norms of the residual forces. A trivial solution is to set all of the kernel values to zeros. To obtain physically meaningful kernels, additional information must be given. For instance, when the kernel for τ_{xy} to describe the equilibrium condition in the x-direction is given, the other two kernels, which are for σₓₓ, and σ_{yy}, can be learned. Similarly, when the kernel for τ_{xy} to describe the equilibrium condition in they-direction is given, the other two kernels can be learned. The kernels learned by the DNN are shown in FIG. 2B and are almost identical to those derived mathematically shown in FIG. 2A. The results show that the kernels encoded in the DNN to describe the conditions for equilibrium are correct, as the same kernels can be learned from the hidden correlation between the elasticity and strain distributions by the DNN.

A strain distribution alone does not provide sufficient information to generate a unique elasticity distribution; an additional physical constraint must be imposed. To develop the process, a first investigation of the effect of physical constraints on prediction accuracy must be done. This demonstrate that the embodiments can generate accurate predictions when imposing a proper physical constraint based on either the total applied force on the boundary or the mean elasticity. In practice, measured strains naturally contain a certain amount of noise. This led to an investigation into the effect of noise in measurements on prediction accuracy. This shows that the embodiments are robust when dealing with noisy measurements. To evaluate the performance of the embodiments, it was compared to OpenQSEI, an iterative model-based elastography method using FEM. The results show that the embodiments generate more accurate predictions and is computationally more efficient compared with OpenQSEI.

Most elastography problems assume that measurements are known everywhere in a body. Here, the embodiments consider a more challenging problem in which some of the measurements in a body are missing. Noisy measurements may still provide useful information for learning the hidden elasticity. However, missing measurements not only provide no information for solving the inverse problem but also may cause the calculation to break down. The embodiments consider a uniform body containing a soft inclusion with a shape similar to the University of California, Berkeley “Cal” logo. In this “Cal” model, the modulus values of the body and soft inclusion are 1 MPa and 0.1 MPa, respectively.

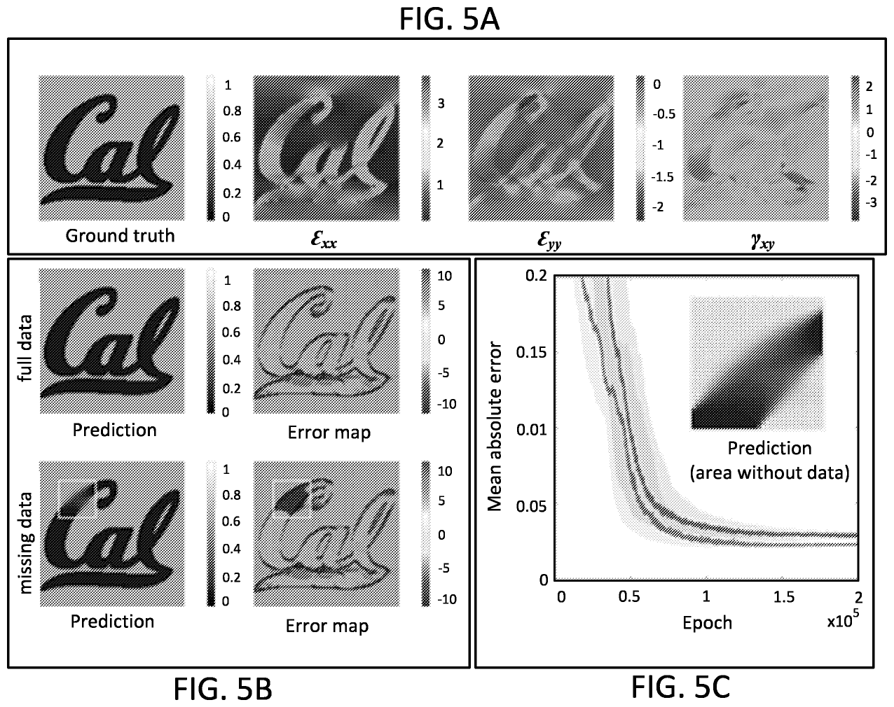

The elasticity and strain distributions of the model are shown in FIG. 5A. In the first scenario, it is assumed that there is no missing data in the measured strains. In the second scenario, it is assumed that the measured strains in an arbitrary area are missing (set to zeros). The predicted elasticity distributions and relative error maps, compared with the ground-truth elasticity distribution, are shown in FIG. 5B, and the error over the training epochs is shown in FIG. 5C.

In the first scenario, the predicted elasticity distribution is accurate with a mean relative error (MRE) of 3.01%. Larger errors occur on the boundary between the body and soft inclusion due to large elasticity differences. In the second scenario, the predicted elasticity distribution is still accurate with an MRE of 6.97%, given that the measurements on a squared area (corresponds to 6.25% of the total area) are missing, shown in the boxed area in FIG. 5B. Larger errors occur on the boundary between the body and soft inclusion and also in the area without data. If the area without data is excluded, the MRE is reduced to 2.95%, which is almost the same as the MRE observed in the first scenario with the full data (3.01%).

The results show that, for the embodiments, missing measurements only reduce the prediction accuracy in the area without data but do not affect the prediction accuracy in the other areas. Moreover, the DNN is trained to learn the hidden elasticity as a function of positions (FIG. 1). Once such a function is learned, the embodiments can predict a probable elasticity distribution for the area even without measurements, shown in inner figure of FIG. 5C. Lastly, the discussion compares the embodiments to DeepFill, one of the state-of-the-art image inpainting methods. DeepFill is based on a variant of generative adversarial networks, named SN-PatchGAN, with gated convolution trained with millions of images. DeepFill is used to fill missing pixels of predicted elasticity images generated by the embodiments and show that the results obtained from both methods are of equivalent quality

For other elastography methods, the resolution of predicted elasticity distributions, or elasticity images, depends on the resolution of measurements. The methods of the embodiments, in theory, can generate elasticity images with any resolution. Here, the embodiments apply the resulting architecture to generate a high-resolution elasticity image from low resolution measurements.

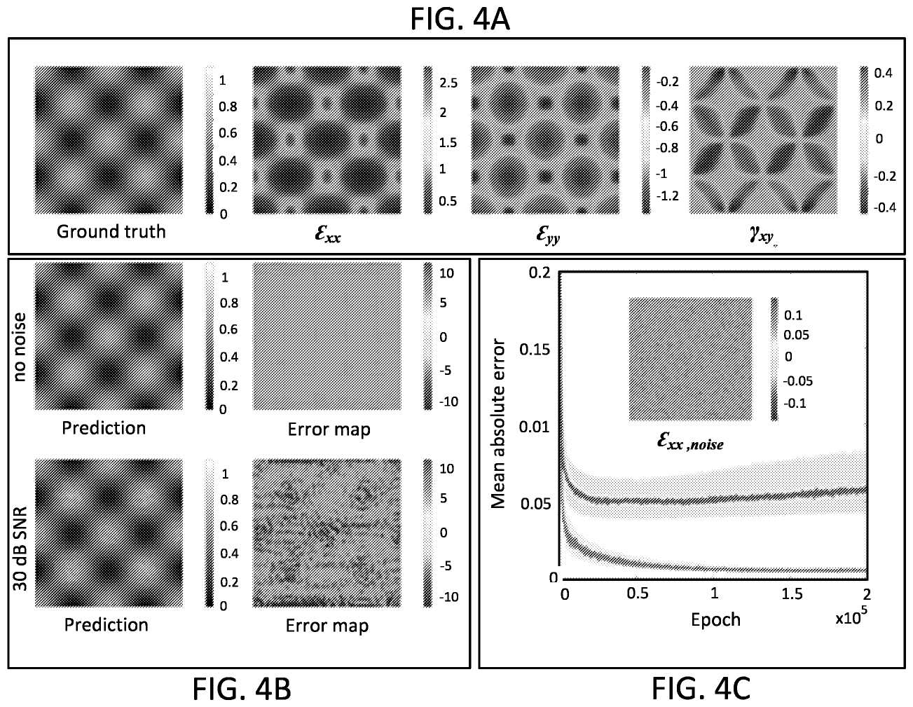

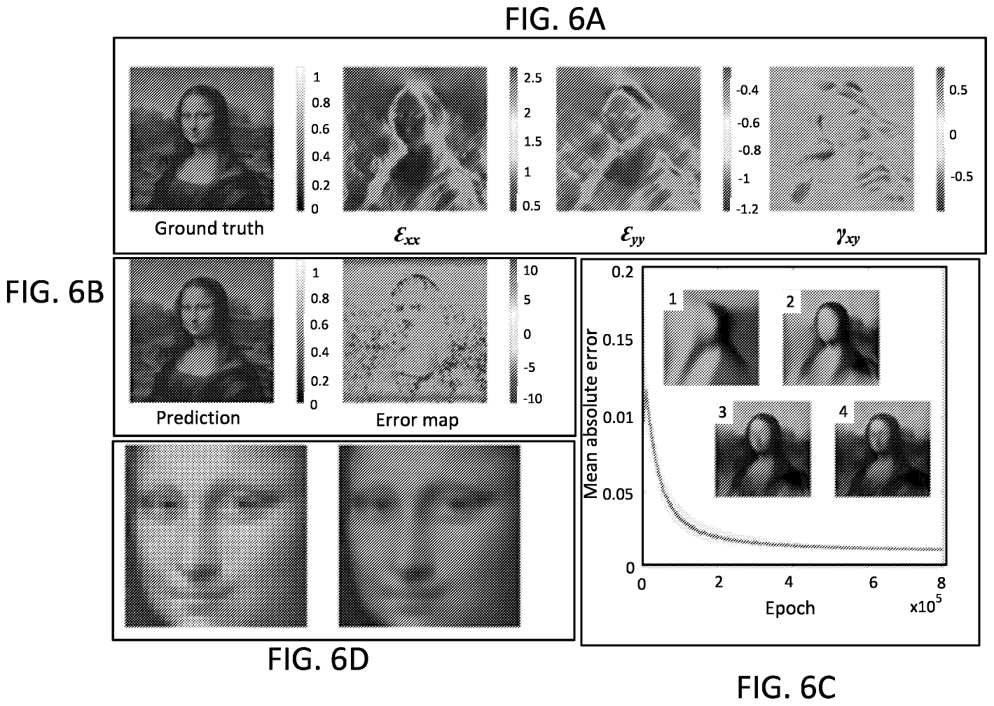

The process considers an inhomogeneous body with an elasticity distribution based on the Mona Lisa by Leonardo da Vinci. In this “Mona Lisa” model, the maximum and minimum Young's moduli are set to be 1 MPa and 0.1 MPa, respectively. The elasticity and strain distributions of the model are shown in FIG. 6A. The predicted elasticity distribution and relative error map are shown in FIG. 6B, and the error over the training epochs is shown in FIG. 6C. While the Mona Lisa model has an extremely complex elasticity distribution, the prediction accuracy is high with an MRE of 2.73%. No visible difference between the ground truth and prediction can be seen. To understand how the embodiments learn the hidden elasticity, intermediate predictions generated during the learning process are shown in the inner figures of FIG. 6C. Interestingly, it can be seen that the embodiments draw an outline first and then adds more details gradually. This process is similar to how an artist draws. The Mona Lisa model is discretized with a 128×128 mesh. The resolution of the measured strains is 128×128 (FIG. 4A). After learning the hidden elasticity from the measured strains, the embodiments can generate elasticity images of arbitrary resolution. Here, the embodiments are applied to generate an elasticity image of a higher resolution-512×512. For comparison, a crop of the ground-truth image and that of the super-resolution image are shown in FIG. 6D. The super-resolution image seems realistic and provides more details compared with the ground-truth image.

A conventional deep-learning model is trained on a data set and can be applied to predict an elasticity distribution based on a new strain distribution without retraining the model. The embodiments, on the other hand, is not supervised by labeled data, and therefore its performance is not limited by the amount, distribution, and accuracy of the data. However, the embodiments need to be retrained for different elastography problems. Therefore, the computational efficiency of the embodiments is essential for elasticity imaging in practice. To make an accurate prediction for the Mona Lisa model shown FIG. 6B, the embodiments take about 80 min for training 800,000 epochs on a workstation using a single graphics processing unit (GPU). After about 3,000 epochs (˜20 s) of training, an intermediate prediction with much detail can already be obtained, shown in the inner figures of FIG. 6C. Lastly, the embodiments were compared with ESRGAN, which won first place in the 2018 Perceptual Image Restoration and Manipulation Challenge on Perceptual Image Super-resolution. Applying ESRGAN to produce a high-resolution version of a predicted elasticity image generated by ElastNet and show that the results obtained from both methods are comparable.

The above discussion demonstrates that the method of the embodiments can accurately learn the hidden elasticity of a body from a given strain distribution. The prediction accuracy depends on the complexity of the hidden elasticity. A higher prediction accuracy may be expected when the elasticity distribution is simpler or smoother. To make an accurate prediction on the hidden elasticity of the Mona Lisa model in FIG. 6B, the proposed method takes about 80 minutes for running 800,000 epochs on a single NVIDIA Tesla V100 GPU. However, after about 3,000 epochs (20 seconds) of training, an intermediate prediction with enough details can already be obtained, shown in the inner figures of FIG. 6C. With further improvements, the proposed method may have the potential to be used in an environment requiring real-time elasticity imaging.

As mentioned above, the discussion now provides information on finite element analysis. A body of interest is discretized by four-node quadrilateral elements with a 128×128 mesh. The modulus of each element is determined based on the elasticity distribution of the body. To simulate an incompressible material, the Poisson's ratio of each element is set to 0.5. The top and bottom boundaries are free. The left boundary is fixed in such a way that the movements are not allowed along the horizontal direction (x-direction) but are allowed along the vertical direction (y-direction). To obtain a nonzero strain distribution of the body, external displacements along the x-direction are applied on the right boundary. The applied displacements are 1% of the body length. An average normal strain along the x-direction (εₓₓ) of 1% can be generated. The strain distribution is calculated from the displacement distribution.

Turning to the encoded domain knowledge of elasticity, for a two-dimensional body, the relation between the strain and displacement with respect to the Cartesian axes is given by:

$$ε = \{ \begin{matrix}{ε}_{xx} \\ {ε}_{yy} \\ {γ}_{xy}\end{matrix} \} = \{ \begin{matrix}\frac{\partial u}{\partial x} \\ \frac{\partial v}{\partial y} \\ \frac{\partial u}{\partial y} + \frac{\partial v}{\partial x}\end{matrix} \} \qquad ( 5 )$$

where ε is the strain vector, u and v are the horizontal and vertical component of the displacement, respectively. The constitutive elasticity relation for a linear elastic isotropic material in plane stress is given by:

$$σ = \{ \begin{matrix}{σ}_{xx} \\ {σ}_{yy} \\ {τ}_{xy}\end{matrix} \} = \frac{E}{1 - {v}^{2}} [ \begin{matrix}1 & v & 0 \\ v & 1 & 0 \\ 0 & 0 & ( 1 - v ) / 2\end{matrix} ] \{ \begin{matrix}{ε}_{xx} \\ {ε}_{yy} \\ {γ}_{xy}\end{matrix} \} \qquad ( 6 )$$

where σ is the stress vector, E is the Young's modulus, and v is the Poisson's ratio. Here, the Poisson's ratio is set to 0.5 for incompressible materials. The conservation of linear momentum is typically written in a differential form:

$$\frac{\partial {σ}_{xx}}{\partial x} + \frac{\partial {τ}_{yx}}{\partial y} = 0 \\ \frac{\partial {σ}_{yy}}{\partial y} + \frac{\partial {τ}_{xy}}{\partial x} = 0 \qquad ( 7 )$$

These PDEs carry the derivatives of the stress field, which are the functions of the derivatives of the strain field and elasticity field. The derivatives of the strain field may be calculated from measured strains. However, measured strains naturally contain noise and the calculation of the derivatives can amplify the noise significantly. The derivatives of the elasticity field cannot be calculated accurately when the derivatives of the strain field are inaccurate. To mitigate this potential problem, one can rewrite the conservation of linear momentum in a finite difference form:

$$\frac{{σ}_{xx} ( x + Δ x , y ) - {σ}_{xx} ( x , y )}{Δ x} + \frac{{τ}_{yx} ( x , y + Δ y ) - {τ}_{yx} ( x , y )}{Δ y} = 0 \\ \frac{{σ}_{yy} ( x , y + Δ y ) - {σ}_{yy} ( x , y )}{Δ y} + \frac{{τ}_{xy} ( x + Δ x , y ) - {τ}_{xy} ( x , y )}{Δ x} = 0 \qquad ( 8 )$$

From these equations, the equilibrium conditions for a small cube with sides of length h can be expressed in Equation 1.

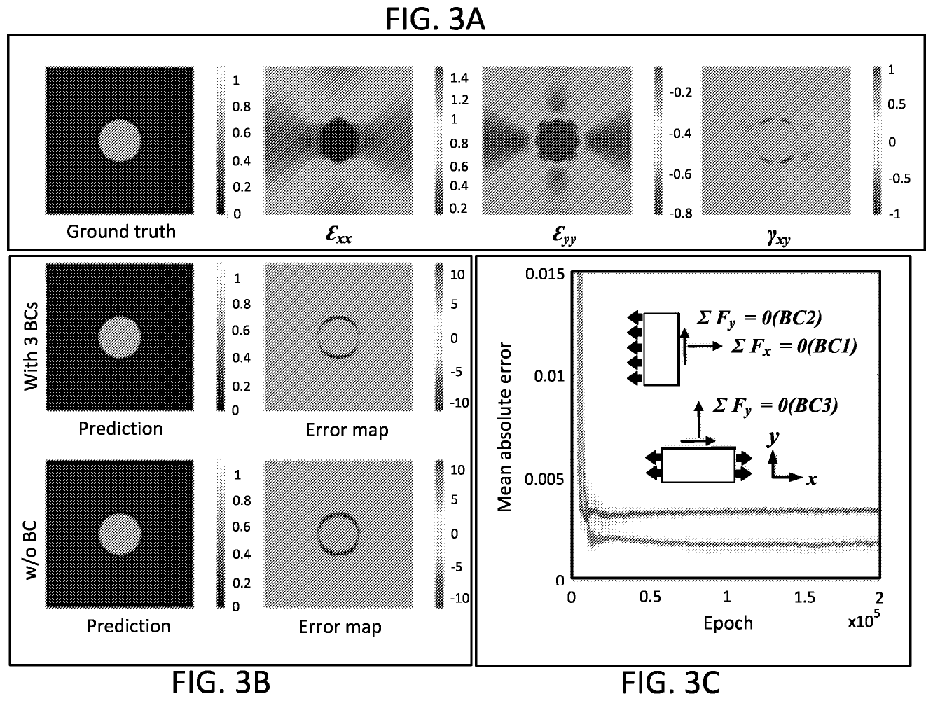

The error over training, shown in FIGS. 3C, 4C, 5C, and 6C, is quantified by the mean absolute error (MAE), defined as

$$MAE = \frac{1}{{n}^{2}} {\sum }_{1}^{n} {\sum }_{j}^{n} {error}_{absolute} ( i , j ) , \qquad ( 9 )$$

where n is the dimension of the elasticity image in both x-direction and y-direction error_{absolute} is the absolute error of a prediction at each material point, defined as:

error_{absolute}(i,j)=|E_{pred}−Eₜᵣᵤₜₕ(i,j)|, (10)

where E_{pred} is the predicted elasticity, and Eₜᵣᵤₜₕ is the ground-truth elasticity.

The MAE can be used to compare the performances of different learning algorithms on the same model. However, the MAE may not be an ideal quantity to compare the accuracies between different models. A larger MAE can be expected when the mean elasticity of the model is larger. Therefore, one can use the MRE to compare the accuracies between different models. The MRE is defined as:

$$MRE = \frac{1}{{n}^{2}} {\sum }_{i}^{n} {\sum }_{j}^{n} \vert {error}_{relative} ( i , j ) \vert , \qquad ( 11 )$$

where errorᵣₑₗₐₜᵢᵥₑ(i, j) (unit: %) is the relative error of a prediction at each material point, defined as:

$${error}_{relative} ( i , j ) = 100 \times {\sum }_{i}^{m} {\sum }_{j}^{m} ( {E}_{pred} ( i , j ) - {E}_{truth} ( i , j ) ) / {E}_{truth} ( i , j ) . \qquad ( 12 )$$

The relative error is used to generate the error maps show in FIGS. 3B, 4B, 5B, and 6B.

The DNN consists of 16 fully connected hidden layers with 128 neurons per layer. The rectified linear unit is adopted as the activation function. The input of the DNN is a vector of two variables (x, y) representing the position p of a material point, and the output is the elasticity E of the point. The stress σ at a material point is calculated by the elastic constitutive relation based on the measured strain ε and predicted elasticity E. Full-batch learning is used when training the DNN, meaning the entire training data set is processed and the error is accumulated. A predicted stress distribution σ is generated when the entire data set is passed forward through the DNN. A convolutional layer consisting of 6 filters of kernel size 3×3 with stride 1 is used to generate residual force maps. In these 6 filters, 3 of them are encoded for evaluating the equilibrium condition in the x-direction and the other 3 are for the y-direction as shown in FIG. 2A.

An Adam (adaptive moment estimation) optimizer may be adopted to minimize the loss function of the DNN. The loss function consists of two parts: one is from the residual forces and the other is from a physical constraint. The residual forces can be measured by the MAE. However, larger residual forces can be expected in areas with larger elasticity. Therefore, the accuracy (relative error) in areas with smaller elasticity will be compromised when using the MAE to measure the residual forces. To make the relative error maps more uniform, the normalized MAE is adopted to measure the residual forces when training the DNN to learn the hidden elasticity. The normalized MAE is defined as:

$${loss}_{force} = \frac{1}{{m}^{2}} {\sum }_{i}^{m} {\sum }_{j}^{m} \frac{\vert e ( i , j ) \vert}{\hat{{E}_{pred}} ( i , j )} , \qquad ( 13 )$$

where m is the dimension of the residual force map in both x-direction and the y-direction, e is the residual force, and Ê_{pred} is the sum of the predicted elasticity values in a cube containing 3×3 material points. Ê_{pred} is calculated as:

Ê_{pred}(i,j)=Σ_{α=1}³Σ_{b=1}³w_{E}(a,b)E_{pred}(i+α−1,j+b−1) (14)

where w_{E} is a convolution kernel of ones. Two types of physical constraints are considered. One is based on the total applied force on the boundary, in which three force boundary conditions (BCs) can be added to the loss function. The penalty terms for these BCs are defined as:

$${loss}_{BC 1} = {\sum }_{i}^{m} \vert ( {\sum }_{j}^{m} {σ}_{xx} ( i , j , ) ht ) - F \vert \\ {loss}_{BC 2} = {\sum }_{i}^{m} \vert {\sum }_{j}^{m} {τ}_{xy} ( i , j , ) ht \vert \\ {loss}_{BC 3} = {\sum }_{i}^{m} \vert {\sum }_{j}^{m} {σ}_{yy} ( i , j , ) ht \vert \qquad ( 15 )$$

where F is the total applied force on the right boundary, which is the same as the total reaction force (along the x-direction) on the left boundary. These penalty terms can be applied to constrain the distribution of the internal stresses. The other type of physical constraints is based on the mean elasticity. The penalty term for the mean elasticity is defined as:

$${loss}_{elast} = \vert \frac{1}{{n}^{2}} {\sum }_{i}^{n} {\sum }_{j}^{n} {E}_{pred} ( i , j ) - \frac{1}{{n}^{2}} {\sum }_{i}^{n} {\sum }_{j}^{n} {E}_{truth} ( i , j ) \vert \qquad ( 16 )$$

For the inclusion model, shown in FIG. 3, sinusoidal model, shown in FIG. 4, and the Cal model, shown in FIG. 5, the DNN is trained for 200,000 epochs. For the Mona Lisa model, shown in FIG. 6, due to the extremely complex hidden elasticity, the DNN is trained for 800,000 epochs. As the weights in the DNN are randomly initialized before training, the predicted elasticity distribution cannot be exactly the same when training with different initial weights. To better evaluate the performance of the system, the predictions reported here are the average values after training the DNN 100 times with different initial weights. The DNN is trained using TensorFlow with a single GPU, such as an NVIDIA Tesla V100 or Titan V.

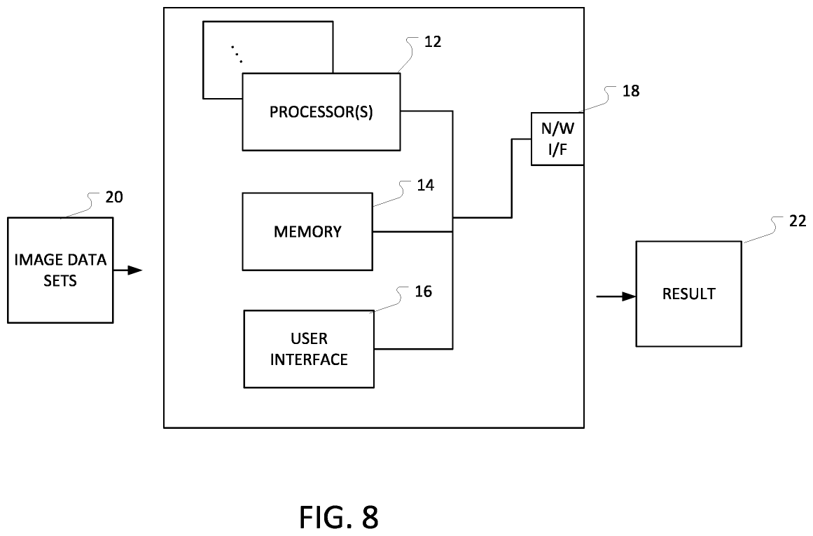

Referring back to FIG. 1, the neural network may comprise a single or multiple GPUs executing code that causes the one or more processor to perform the methods of the embodiments discussed here. However, it may be an advantage for the system to use only one processor, such as that mentioned above. FIG. 8 show an embodiment of such a system. The processor or processors 12 “contain” the neural network, meaning it has been programmed to operate as a neural network, a memory 14 to contain operating instructions for the processor(s), and a user interface 16. The user interface may include such things as a display, which may comprise a touch screen, user input devices such as mice and keyboards, etc. The computing system in which the processors are contained may have a network interface 18. The system receives the image or images as data sets 20 and produces the result discussed above 22.

Aspects of the disclosure may operate on a particularly created hardware, on firmware, digital signal processors, or on a specially programmed general purpose computer including a processor operating according to programmed instructions. The terms controller or processor as used herein are intended to include microprocessors, microcomputers, Application Specific Integrated Circuits (ASICs), and dedicated hardware controllers. One or more aspects of the disclosure may be embodied in computer-usable data and computer-executable instructions, such as in one or more program modules, executed by one or more computers (including monitoring modules), or other devices. Generally, program modules include routines, programs, objects, components, data structures, etc. that perform particular tasks or implement particular abstract data types when executed by a processor in a computer or other device. The computer executable instructions may be stored on a non-transitory computer readable medium such as a hard disk, optical disk, removable storage media, solid state memory, Random Access Memory (RAM), etc. As will be appreciated by one of skill in the art, the functionality of the program modules may be combined or distributed as desired in various aspects. In addition, the functionality may be embodied in whole or in part in firmware or hardware equivalents such as integrated circuits, FPGA, and the like. Particular data structures may be used to more effectively implement one or more aspects of the disclosure, and such data structures are contemplated within the scope of computer executable instructions and computer-usable data described herein.

The disclosed aspects may be implemented, in some cases, in hardware, firmware, software, or any combination thereof. The disclosed aspects may also be implemented as instructions carried by or stored on one or more or non-transitory computer-readable media, which may be read and executed by one or more processors. Such instructions may be referred to as a computer program product. Computer-readable media, as discussed herein, means any media that can be accessed by a computing device. By way of example, and not limitation, computer-readable media may comprise computer storage media and communication media.

Computer storage media means any medium that can be used to store computer-readable information. By way of example, and not limitation, computer storage media may include RAM, ROM, Electrically Erasable Programmable Read-Only Memory (EEPROM), flash memory or other memory technology, Compact Disc Read Only Memory (CD-ROM), Digital Video Disc (DVD), or other optical disk storage, magnetic cassettes, magnetic tape, magnetic disk storage or other magnetic storage devices, and any other volatile or nonvolatile, removable or non-removable media implemented in any technology. Computer storage media excludes signals per se and transitory forms of signal transmission.

Communication media means any media that can be used for the communication of computer-readable information. By way of example, and not limitation, communication media may include coaxial cables, fiber-optic cables, air, or any other media suitable for the communication of electrical, optical, Radio Frequency (RF), infrared, acoustic or other types of signals.

Conventional model-based elastography using FEM represents an elasticity image as a set of pixels. The embodiments here consider an elasticity image as a mathematical function. Given a large enough DNN, any complex elasticity image can be approximated by the DNN. This approach ensures that a target elasticity distribution can be represented as a function and allows a learning algorithm to gradually update the function. Here, all material points in a body of interest are assumed to be linear, isotropic, and incompressible. However, the embodiments can be extended to consider compressible materials if necessary. For instance, instead of setting the Poisson's ratio at each material point to 0.5, it can be represented as an unknown variable. The DNN f_{θ} will take the information of position p at each material point as an input and output its elasticity and Poisson's ratio simultaneously, (E, v)=(f_{θ}(p).

The embodiments show that, by combining the theory of elasticity with a deep-learning approach, they can generate rapid and accurate predictions. The prediction accuracy depends on the complexity of the hidden elasticity. A higher prediction accuracy may be expected when the elasticity distribution is simpler or smoother. The embodiments are also robust when dealing with noisy measurements. For measurements with missing data, only the prediction accuracy in the area without data will be compromised and the prediction accuracy in the other areas will not be affected. Once the function of an elastic image is learned, the DNN can predict probable elasticity distributions for areas even without measurements and generate elasticity images of arbitrary resolution.

Modifications may include incorporating other DNNs specifically trained for image inpainting or single-image super-resolution. With prior knowledge from the theory of elasticity, the embodiments do not require any labeled data for training, and therefore have no artificial constraint imposed on possible elasticity distributions. This advantage allows the embodiments to be applied to a broad range of elastography problems in which no prior knowledge is available on the hidden elasticity.

In this manner, one can determine the material property distribution of solids non-invasively. The ability to measure changes in elasticity over time allows for early detection of diagnoses when the solids are human tissue. The embodiments can learn the hidden elasticity of solids accurately, and enables non-invasive characterization of materials for various applications.

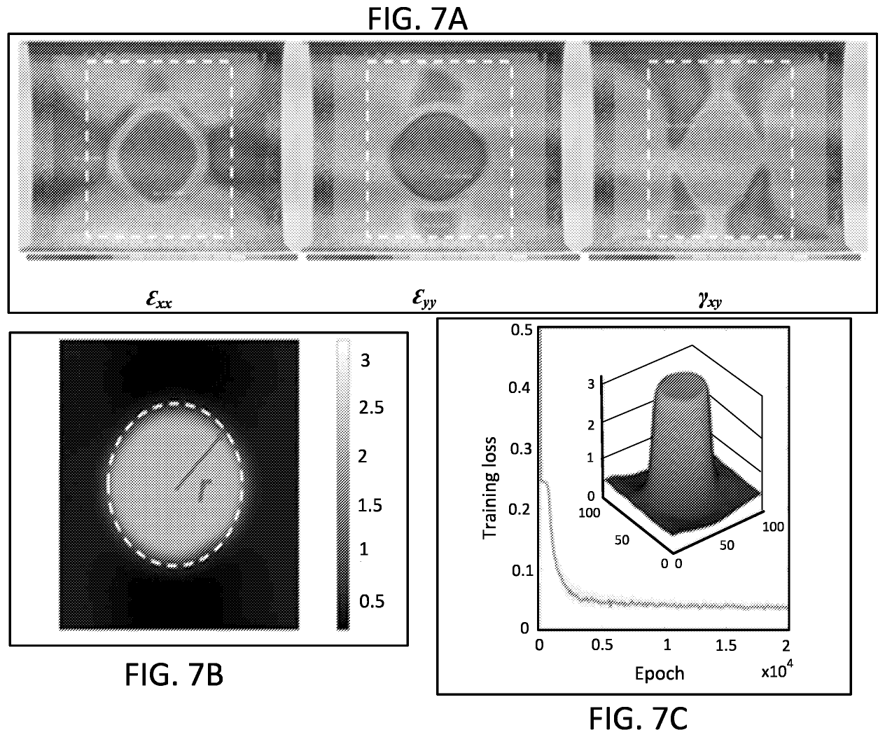

Experimental validation of method and embodiment in FIG. 7 show good correlation between what is predicted and actual from testing. FIG. 7A shows experimental strain images using digital image correlation. FIG. 7B shows predicted elasticity distribution from an embodiment with a similar modulus ration as the experimental value. FIG. 7C represents the loss over the training epochs.

Additionally, this written description makes reference to particular features. It is to be understood that the disclosure in this specification includes all possible combinations of those particular features. For example, where a particular feature is disclosed in the context of a particular aspect, that feature can also be used, to the extent possible, in the context of other aspects.

Also, when reference is made in this application to a method having two or more defined steps or operations, the defined steps or operations can be carried out in any order or simultaneously, unless the context excludes those possibilities.

Although specific embodiments have been illustrated and described for purposes of illustration, it will be understood that various modifications may be made without departing from the spirit and scope of the disclosure. Accordingly, the invention should not be limited except as by the appended claims.

1. A computer-implemented method of predicting elasticity of a solid, comprising:
receiving a data set comprised of position data and corresponding strain data for points on a solid at a deep neural network (DNN);
producing a predicted stress distribution;
applying convolutional filters to the predicted stress distribution to produce residual force maps; and
predicting an elasticity distribution of the solid by iteratively using the residual force maps and an equilibrium condition until the predicted stress distribution satisfies the equilibrium condition, producing the final elasticity distribution.

2. The computer-implemented method of claim 1, further comprising updating weights used in determining the elasticity distribution based upon the strain data at each iteration.

3. The computer-implemented method of claim 1, wherein using the equilibrium data comprises applying filters encoded with an equilibrium condition in an x-direction and a y-direction.

4. The computer-implemented method of claim 1, producing the predicted stress distribution comprises applying an elastic constitutive relation encoded into the DNN prior to receiving the data set.

5. The computer-implemented method of claim 1, further comprising encoded the equilibrium conditions into the DNN prior to receiving the data set.

6. The computer-implemented method of claim 1, wherein the DNN operates using an elastic constitutive relation and equilibrium conditions with no labeled data.

7. The computer-implemented method of claim 1, wherein the strain data comprises strain data for each point of the position data and results from one of either experiments or simulation.

8. The computer-implemented method of claim 1, wherein the final elasticity distribution has a higher resolution than the strain data.

9. The computer-implemented method of claim 1, wherein the solid comprises human tissue.

10. The computer-implemented method of claim 1, further comprising applying an adaptive moment optimizer in the DNN.

11. The computer-implemented method of claim 1, wherein the DNN performs full-batch learning.

12. A computing device, comprising:
one or more processors configured to execute code that will cause the one or more processors to: receive a data set comprised of position data and corresponding strain data for points on a solid at a deep neural network (DNN);
produce a predicted stress distribution;
apply convolutional filters to the predicted stress distribution to produce residual force maps; and
predict an elasticity distribution of the solid by iteratively using the residual force maps and an equilibrium condition until the predicted stress distribution satisfies the equilibrium condition, producing the final elasticity distribution.
